(19) United States
(12) Patent Application Publication
(10) Pub. No.: US 2024/0139084 A1
(43) Pub. Date: May 2, 2024

(54) COSMETIC COMPOSITION COMPRISING SILICA AND LAUROYL LYSINE

(71) Applicant: The Procter & Gamble Company, Cincinnati, OH (US)

(72) Inventors: Fanny Himawan, Singapore (SG); Shuhei Tanaka, Singapore (SG)

(21) Appl. No.: 18/081,753

(22) Filed: Dec. 15, 2022

Related U.S. Application Data
(60) Provisional application No. 63/415,648, filed on Oct. 12, 2022.

(51) Int. Cl. A61K 8/25; A61K 8/02; A61K 8/44; A61K 8/92; A61Q 19/00

(52) U.S. Cl. CPC A61K 8/25; A61K 8/0245; A61K 8/025; A61K 8/442; A61K 8/92; A61Q 19/00; A61K 2800/10; A61K 2800/412

(57) ABSTRACT

A cosmetic composition containing from about 0.1% to about 10% silica, from about 0.1% to about 10% of lauroyl lysine, and water. The silica can have an average particle size of from about 0.5 microns to about 35 microns and an oil absorbance of from about 1 ml/100 g to about 130 ml/100 g. The composition can provide improved sensory feel during and/or after the application to skin, such as reduced heavy coated feel; reduced sticky/tacky feel; and/or reduced friction/draggy feel, while providing moisturized feel, especially in cosmetic compositions containing a higher level of water, and/or in the cosmetic compositions containing lower level of oils.

### FIELD OF THE INVENTION

The present invention is directed to a cosmetic composition, comprising by weight: (a) from about 0.1% to about 10% of silica having an average particle size of from about 0.5 microns to about 35 microns, and having an oil absorbance of from about 1 ml/100 g to about 130 ml/100 g; (b) from about 0.1% to about 10% of lauroyl lysine; (c) from about 55% to about 99% of water. The composition can provide improved sensory feel during and/or after the application to skin, such as: reduced heavy coated feel; reduced sticky/tacky feel; and/or reduced friction/draggy feel, while providing moisturized feel, especially in cosmetic compositions containing a higher level of water, and/or in the cosmetic compositions containing lower level of oils.

### BACKGROUND OF THE INVENTION

Mammalian keratinous tissue, particularly human skin, is subjected to a variety of insults by both extrinsic and intrinsic factors. Such extrinsic factors include ultraviolet radiation, environmental pollution, wind, heat, infrared radiation, low humidity, harsh surfactants, abrasives, etc. Intrinsic factors, on the other hand, include chronological aging and other biochemical changes from within the skin. Whether extrinsic or intrinsic, these factors result in visible signs of skin damage. Typical skin damages in aging or damaged skin include fine lines, wrinkling, hyperpigmentation, sallowness, sagging, dark under-eye circles, puffy eyes, enlarged pores, diminished rate of turnover, and abnormal desquamation or exfoliation. Additional damage incurred as a result of both external and internal factors includes visible dead skin i.e., flaking, scaling, dryness, and roughness.

Currently, there are a number of personal care products that are available to consumers, which are directed toward improving the health and physical appearance of keratinous tissues such as the skin, hair, and nails. The majority of these products are directed to delaying, minimizing or even eliminating skin wrinkling, spots, and other histological changes typically associated with the aging of skin or environmental damage to human skin. Consumers may prefer topically applied products since they are not only effective, but also safe and pleasant to use.

A variety of ingredients are used in such products. However, some of such ingredients may provide undesirable sensory feel during and/or after the application to skin, such as: heavy coated feel, greasy/oily feel, sticky/tacky feel, and/or friction/draggy feel.

For example, French patent application publication No. 3021528 describes that there is a need to find new cosmetic compositions, in particular cosmetic formulations for moisturizing the skin having a suitable texture, in particular a light, non-greasy texture. The French publication discloses a cosmetic formulation comprising: an emulsifier comprising at least one jojoba wax ester, at least one beeswax ester and cetyl alcohol; a particulate cosmetic powder in spherical form with an average particle size of 1 to 25 μm; a cosmetic powder in sheet form with an average particle size of 5 to 40 μm; and optionally further comprising a cosmetic powder in the form of flakes with an average flake diameter of 2 to 60 μm and/or a cosmetic powder in the form of polygonal crystals with an average crystal diameter of 2 to 60 μm. The French publication also discloses in Table 1 a cosmetic composition comprising 3.5% of Shea butter, 0.5% of SUNSIL-150H (having a particle size of 10-15 microns and oil absorbance of 140-210 ml/100 g), 0.4% of AMIHOPE-LL, 0.4% of CELLULOFLAKE and 53.4% of water.

However, it has been found that there still exists a need to provide improved sensory feel during and/or after the application to skin, such as: reduced heavy coated feel; reduced sticky/tacky feel; and/or reduced friction/draggy feel, while providing moisturized feel, especially in cosmetic compositions containing a higher level of water, and/or in the cosmetic compositions containing lower level of oils.

Additionally, there may exists a need for a cosmetic composition, which provides at least one of the followings:
improved sensory feel during and/or after the application to skin, such as: reduced heavy coated feel; reduced sticky/tacky feel; and/or reduced friction/draggy feel, while providing fresh/light feel from the composition containing a higher level of water;
improved sensory feel during and/or after the application to skin, such as: reduced heavy coated feel; reduced sticky/tacky feel; and/or reduced friction/draggy feel, while providing reduced greasy gloss/oily shine from oil from the composition.

### SUMMARY OF THE INVENTION

The present invention is directed to a cosmetic composition comprising by weight: (a) from about 0.1% to about 10% of silica having an average particle size of from about 0.5 microns to about 35 microns, and having an oil absorbance of from about 1 ml/100 g to about 130 ml/100 g; (b) from about 0.1% to about 10% of lauroyl lysine; (c) from about 55% to about 99% of water.

The composition can provide improved sensory feel during and/or after the application to skin, such as: reduced heavy coated feel; reduced sticky/tacky feel; and/or reduced friction/draggy feel, while providing moisturized feel, especially in cosmetic compositions containing a higher level of water, and/or in the cosmetic compositions containing lower level of oils.

It has now been surprisingly found that, by the use of lauroyl lysine and the specific silica, the composition can provide such improved sensory feel by horizontal smooth feel due to the specific silica and vertically bouncy and/or soft feel due to lauroyl lysine, while providing moisturized feel.

### BRIEF DESCRIPTION OF THE DRAWINGS

FIG. 1 illustrates an exemplary test setup for the Tack Method/Test.

### DETAILED DESCRIPTION OF THE INVENTION

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all ingredient percentages are based on the weight of the cosmetic composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive and combinable to form narrower ranges not explicitly disclosed. For example, delineated upper and lower range limits are interchangeable to create further ranges.

The compositions can contain comprise, consist essentially of, or consist of, the essential components as well as optional ingredients described herein. As used herein, “consisting essentially of” means that the composition or component may only include additional ingredients that do not materially alter the basic and novel characteristics of the claimed composition or method. As used in the description and the appended claims, the singular forms “a”, “an”, and “the” are intended to include the plural forms as well, unless the context clearly indicates otherwise.

### Definitions

“About” modifies a particular value by referring to a range equal to plus or minus twenty percent (+/−20%) or less (e.g., less than 15%, 10%, or even less than 5%) of the stated value.
“Apply” or “application”, as used in reference to a composition, means to apply or spread the compositions of the present invention onto a human skin surface such as the epidermis.
“Derivative,” herein, means amide, ether, ester, amino, carboxyl, acetyl, and/or alcohol derivatives of a given compound.
“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein.
“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration, food additives, and materials used in non-cosmetic consumer products including over-the-counter medications.
“Cosmetic composition” means a composition comprising a cosmetic agent. Examples of cosmetic compositions include color cosmetics (e.g., foundations, lipsticks, concealers, and mascaras), skin care compositions (e.g., moisturizers and sunscreens), personal care compositions (e.g., rinse-off and leave on body washes and soaps), hair care compositions (e.g., shampoos and conditioners).
“Skin care” means regulating and/or improving a skin condition (e.g., skin health, appearance, or texture/feel). Some nonlimiting examples of improving a skin condition include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.
“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).
“Skin care composition” means a composition that includes a skin care active and regulates and/or improves skin condition.
“Synergy,” and variations thereof, means that the effect provided by a combination of two or more materials is more than the additive effect expected for these materials.
“Treatment period,” as used herein, means the length of time and/or frequency that a material or composition is applied to a target skin surface.

### Cosmetic Composition

The cosmetic composition can comprise: silica having an average particle size of from about 0.5 microns to about 35 microns and has an oil absorbance of from about 1 ml/100 g to about 130 ml/100 g; a lauroyl lysine; and water.

In view of providing improved sensory feeling, the composition meets at least one of the following conditions (i)-(ii), alternatively the composition meets both of the following conditions (i)-(ii):
(i) wherein the weight ratio between silica and lauroyl lysine is from about 15:1 to about 1:10, alternatively 10:1 to about 1:10, alternatively from about 7:1 to about 1:7, alternatively from about 5:1 to about 1:5;
(ii) wherein the particle size ratio between silica and lauroyl lysine is from about 3:1 to about 1:35, alternatively 1:1 to about 1:35, alternatively from about 1:2 to about 1:25, alternatively from about 1:2 to about 1:20.

The composition has a viscosity of alternatively from about 100 cP to about 300,000 cP, alternatively from about 100 cP to about 200,000 cP, alternatively from about 100 cP to about 100,000 cP, alternatively from about 1000 cP to about 55,000 cP, further alternatively from about 1,000 to about 48,0000, especially when the composition has a higher level of water.

If the composition can contain shea butter, cetyl alcohol, Stearyl alcohol and/or behenyl alcohol, and in some examples the composition can contain a limited amount of such shea butter, cetyl alcohol, stearyl alcohol and behenyl alcohol, for providing fresh/light feel from the composition containing a higher level of water. Total level of shea butter, cetyl, stearyl, and behenyl alcohols in the composition is up to about 0.5%, alternatively up to about 0.3%, alternatively up to about 0.1%, alternatively 0%.

The cosmetic compositions herein are intended for topical application to human skin. The compositions herein may optionally include one or more additional skin actives or other ingredients of the type commonly included in topical cosmetic compositions.

The cosmetic compositions herein may be cosmetic compositions, pharmaceutical compositions, or cosmeceutical compositions, and may be provided in various product forms, including, but not limited to, solutions, suspensions, lotions, gels, toners, cleansing liquid washes hydrogels, film-forming products, and the like. In some instances, the composition form may follow from the particular dermatologically acceptable carrier chosen. For example, the composition (and carrier) may be provided in the form of an emulsion (e.g., water-in-oil, oil-in-water, or water-in-oil-in water) or an aqueous dispersion. The cosmetic composition can be in the form of an oil-in-water emulsion.

### Silica

The composition can comprise a silica at a level of from about 0.1% to about 10%, alternatively from about 0.3% to about 7%, alternatively from about 0.5% to about 5%.

The silica useful herein has an average particle size of from about 0.5 microns to about 35 microns, alternatively from about 1 micron to 31 microns, alternatively from about 1 micron to 15 microns, alternatively from about 1 microns to about 10 microns, in view of improved sensory feeling, as too small particles may not provide enough smoothness and too big particles may provide scrubbing-like feeling. The silica can have a spherical shape.

The silica useful herein has an oil absorbance of from about 1 ml/100 g to about 130 ml/100 g, alternatively from about 1 ml/100 g to about 120 ml/100 g, alternatively from about 1 ml/100 g to about 100 ml/100 g, alternatively from about 1 ml/100 g to about 50 ml/100 g, in view of providing a balance between improved sensory feel and moisturized feel

Commercial examples of spherical silicas having such particle size and such oil absorbance are, for example, Goddball G6C having a mean particle size of 3-5 microns and an oil absorbance of 30 ml/100 g, and Goddball E90C having a mean particle size of 30 microns and an oil absorbance of 120 ml/100 g.

### Lauroyl Lysine

The composition can comprise lauroyl lysine at a level of from about 0.1% to about 10%, alternatively from about 0.3% to about 7%, alternatively from about 0.5% to about 5%.

The shape of the lauroyl lysine can be anything. The shape of the lauroyl lysine can be spherical or flat polygonal (selected from flat pentagonal, flat hexagonal, and flat heptagonal, preferably flat hexagonal shape). Alternatively, the shape of the lauroyl lysine is flat polygonal.

The lauroyl lysine in the flat polygonal shape can have a mean particle size of from about 10 microns to about 40 microns, alternatively from about 15 microns to about 35 microns, alternatively from about 20 microns to 30 microns, in view of improved sensory feeling, as too small particles may not provide enough bouncy/soft feel and too big particles may provide scrubbing-like feeling.

Commercial example of such lauroyl lysine in the flat polygonal shape having such particle size is, for example, Amihope LL available from Ajinomoto, having a flat hexagonal shape and having a mean particle size of 20-30 microns.

The lauroyl lysine in spherical shape can have a mean particle size of from about 1 micron to about 30 microns, alternatively from about 2 microns to about 20 microns, alternatively from about 3 microns to 8 microns.

Lauroyl lysine, especially when it's in flat polygonal shape, may be dispersed in the composition as a layered structure comprising some or several flat polygonal crystals. It is believed that the lauroyl lysine provides improved moisturizing/cushioning feel, compared to starch solid powders, cellulose solid powders.

Also, compared to hydrophilic solid polymeric powders such as starch solid powders and cellulose solid powders, it is believed that the lauroyl lysine provides reduced pilling and/or clumping especially when the composition contains higher levels of hydrophilic thickeners. For such reduced pilling and/or clumping, it may be preferred that the composition is substantially free of such hydrophilic solid polymeric powders, i.e., contains 0.1% or less, alternatively 0.05% or less of such hydrophilic solid polymeric powders. The composition can be free of such hydrophilic solid powders, i.e., contains 0% of such hydrophilic solid polymeric powders.

It is also believed the lauroyl lysine may provide matte appearance and/or reduced visibility of skin pores.

### Substantially Free of Microplastic Solid Particulates

The composition can contain silica and lauroyl lysine can provide improved sensory feeling, without the use of microplastic solid particulates. Thus, the composition may also provide environmental benefit in view of the reduction of the use of microplastic solid particulates. Such microplastic solid particulates are, for example, nylon powder, polyurethane powder, polyethylene powder, silicone resin powder. In the present invention, the composition can be substantially free of such microplastic solid particulates. The composition can be is free of such microplastic solid particulates, i.e., contains 0% of such microplastic solid particulates.

### Humectant

The composition can comprise a humectant at a level of from about 0.5% to about 20%, alternatively from about 1% to about 15%, alternatively from about 2% to about 15%, alternatively form about 4% to about 12% by weight of the composition.

Humectants useful herein are polyhydric alcohols intended for moisturizing, reducing scaling and stimulating removal of built-up scale from the skin. Polyhydric alcohols useful herein include, for example, alkylene polyols and their derivatives, polyalkylene glycols. Polyhydric alcohols useful herein are, for example, propylene glycol, dipropylene glycol, polypropylene glycol, polyethylene glycol, sorbitol, hydroxypropyl sorbitol, hexylene glycol, pentylene glycol, 1,3-butylene glycol, 1,2,6-hexanetriol, glycerin, ethoxylated glycerin, propoxylated glycerin and mixtures thereof. The humectants can be glycerin, pentylene glycol, 1,3-butylene glycol, propylene glycol. Alternatively, the humectant is glycerin, especially when using with Vitamin B₃ compounds at a higher level which may cause draggy feel during application and/or reduced moisturized after application.

### Aqueous Carrier/Water

The composition can comprise an aqueous carrier. The aqueous carrier can be substantially water and can be deionized water. Water from natural sources including mineral cations can also be used, depending on the desired characteristic of the composition.

The composition can comprise water at a level of from about 55% to about 99%, alternatively from about 60% to about 98%, alternatively from about 60% to about 95%, alternatively from about 60% to about 90%, by weight of the composition.

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils).

### pH of the Composition

Typically, cosmetic compositions are formulated to have a slightly acidic to neutral pH (i.e., 5.0-7.0), which is believed to improve the stability of certain ingredients in the composition (e.g., niacinamide, salicylates, and neutralized thickeners). However, formulating a skin care composition at low pH (e.g., 2.0-5.0) may also provide certain benefits such as improving the appearance of skin, bolstering the acid mantle of the skin, exfoliating the skin, improving skin texture, and/or providing flexibility in product formulation.

The composition may contain have a lower pH, i.e., pH of less than 5.0, alternatively from about 1.5 to about 4.9, alternatively from about 2.0 to about 4.5, alternatively from about 2.5 to about 4.0, or alternatively from about 3.5 to about 4.0. It has been found that polyacrylate crosspolymer-6 (such as that supplied as “Sepimax Zen” from Seppic) can provide suitable tolerance to low pH environments and the desired feel and opacity properties to the composition.

### Skin Conditioning Agent

Optionally, the composition can contain further comprise a skin conditioning agent. These agents may be selected from humectants and emollients. The amount of skin-condition agent may range from about 1% to about 50%, alternatively from about 2% to about 40%, alternatively from about 5% to about 30%, by weight of the composition.

Humectants are polyhydric alcohols intended for moisturizing, reducing scaling and stimulating removal of built-up scale from the skin. Typical polyhydric alcohols include polyalkylene glycols and alternatively alkylene polyols and their derivatives. Illustrative are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof. The humectant can be glycerin.

When the conditioning agent is an emollient it may be selected from hydrocarbons, fatty acids, fatty alcohols and esters.

### Fatty Alcohol

The compositions herein may include a fatty alcohol. Fatty alcohols refer to high-molecular-weight, straight-chain primary alcohols that have the general structure:

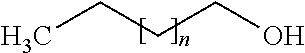

where n=8 to 32.

Fatty alcohols may be natural or synthetic, saturated or unsaturated, branched or straight-chain. Some nonlimiting examples of fatty alcohols commonly used in cosmetic compositions include caprylic, capryl, lauryl, myristyl, cetyl, stearyl, and behenyl alcohols. The fatty alcohols herein may be referred to generically by the number of carbon atoms in the molecule. For example, a “C12 alcohol” refers to an alcohol that has 12 carbon atoms in its chain (i.e., dodecanol).

The fatty alcohol may be included in the compositions herein at 0.0001% to 15% (e.g., 0.0002% to 10%, 0.001% to 15%, 0.025% to 10%, 0.05% to 7%, 0.05% to 5%, or even 0.1% to 3%) by weight of the composition.

### Whitening Agents

The present compositions may contain a whitening agent. The whitening agent useful herein refers to active ingredients that not only alter the appearance of the skin, but further improve hyperpigmentation as compared to pre-treatment. Useful whitening agents useful herein include ascorbic acid compounds, vitamin B 3 compounds, azelaic acid, butyl hydroxy anisole, gallic acid and its derivatives, hydroquinoine, kojic acid, arbutin, mulberry extract, tetrahydrocurcumin, and mixtures thereof. Use of combinations of whitening agents is also believed to be advantageous in that they may provide whitening benefit through different mechanisms.

When used, the compositions can contain from about 0.1% to about 10%, alternatively from about 0.2% to about 5%, by weight of the composition, of a whitening agent.

Ascorbic acid compounds are useful whitening agents and ascorbyl glucoside can be a preferred derivative of the ascorbic acid compounds.

### Conditioning Agents

The compositions herein may include 0.1% to 50% by weight of a conditioning agent (e.g., 0.5% to 30%, 1% to 20%, or even 2% to 15%). Adding a conditioning agent can help provide the composition with desirable feel properties (e.g., a silky, lubricious feel upon application). Some non-limiting examples of conditioning agents include, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof.

Particularly suitable examples of conditioning agents include non-volatile silicone fluids, such as dimethicone copolyol, dimethylpolysiloxane, diethylpolysiloxane, mixed C1-30 alkyl polysiloxanes, phenyl dimethicone, dimethiconol, dimethicone, dimethiconol, silicone crosspolymers, and combinations thereof. Dimethicone may be especially suitable, since some consumers associate the feel properties provided by certain dimethicone fluids with good moisturization.

### Hydrophilic Thickener

In some examples, the compositions may include one or more hydrophilic thickener. When present, the composition can include from about 0.01% to about 5%, alternatively from about 0.1% to about 4%, and alternatively from about 0.1% to about 3%, by weight of the composition of the hydrophilic thickeners.

The hydrophilic thickener useful herein is not particularly limited as long as it is one that is normally used in cosmetic products. Examples include natural or semi-synthetic water-soluble polymers, synthetic water-soluble polymers and inorganic water-soluble polymers.

As the natural or semi-synthetic water-soluble polymers, polysaccharides and derivatives thereof (including water-soluble alkyl-substituted polysaccharide derivatives) can be used. Specific examples include plant-based polymers such as gum arabic, tragacanth gum, galaetan, guar gum, carob gum, karaya gum, carrageenan, pectin, agar, quince seed (marmelo), algecolloid (phaeophyceae extract), starch (rice, corn, potato, wheat) and glycyrrhizinic acid; microbe-based polymers such as xanthan gum, dextran, succinoglycan and pullulan; starch-based polymers such as carboxymethyl starch and methylhydroxypropyl starch; cellulose-based polymers such as methyl cellulose, nitrocellulose, ethyl cellulose, methyl hydroxypropyl cellulose, hydroxyethyl cellulose, sodium cellulose sulfate, hydroxypropyl cellulose, sodium carboxymethyl cellulose (CMC), crystalline cellulose and cellulose powder; and alginic acid-based polymers such as sodium alginate and propylene glycol esters of alginic acid.

The synthetic water-soluble polymers include ionic or non-ionic water-soluble polymers, for example, vinyl-based polymers such as polyvinyl alcohol, polyvinyl methyl ether, polyvinyl pyrrolidone and carboxyvinyl polymers (carbomers); acryl-based polymers such as sodium polyacrylate, poly ethyl acrylate, polyacrylamide compounds and acrylic acid/alkyl methacrylate copolymers (product name “pemulen TR-1”).

The polyacrylamide compounds particularly include polyacrylamide compounds consisting of homopolymers, copolymers or crosspolymers containing one or more constituent units chosen from among 2-acrylamido-2-methylpropane sulfonic acid (hereinafter sometimes abbreviated to “AMPS”), acrylic acid and derivatives thereof.

Specific examples of such polyacrylamide compounds include vinylpyrrolidone/2-acrylamido-2-methylpropane sulfonic acid (salt) copolymers, dimethylacrylarnide/2-acrylamido-2-methylpropane sulfonic acid (salt) copolymers, acrylamide/2-acrylamiclo-2-methylpropane sulfonic acid copolymers, dimethylacrylainide/2-acrylamido-2-methylpropane sulfonic acid crosspolymers crosslinked with methylenebisacrylamide, mixtures of polyacrylamide and sodium polyacrylate, sodium acrylate/2-acrylamido-2-methylpropane sulfonic acid copolymers, hydroxyethyl acrylate/2-acrylamido-2-methylpropane sulfonic acid (salt) copolymers, ammonium polyacrylate, polyacrylamide/ammonium acrylate copolymers, and acrylamide/sodium acrylate copolymers. However, the compounds are not limited to these examples.

Examples of salts in the previous paragraph can include alkali metal salts (such as calcium salts and magnesium salts), ammonium salts, organic amine salts (such as monoethanolamine salts, diethanolamine salts, and triethanolamine salts). One or more of these polyacrylamide compounds may be used.

These polyacrylamide compounds may be synthesized or obtained as commercial products. For example, the vinyl pyrrolidone/2-acrylarnido-2-methylpropane sulfonic acid (salt) copolymer may be “Aristoflex AVC” (manufactured by Clariant), the sodium alylate/2-acrylamido-2-methylpropane sulfonic acid (salt) copolymer may be “Simulgel EG” (manufactured by Seppic) or “Simulgel EPG” (manufactured by Seppic), the acrylamide/2-acrylamido-2-methylpropane sulfonic acid sodium salt copolymer may be “Simulgel 600” (manufactured by Seppic), the acrylamide/2-acrylamido-2-methylpropane sulfonic acid (salt) may be “Sepigel 305” (manufactured by Seppic) or “Sepigel 501” (manufactured by Seppic), the homopolymer of a 2-acrylamido-2-methylpropane sulfonic acid sodium salt may be “1-lostacerin AMPS” (manufactured by Clariant) or “Simulgel 800” (manufactured by Seppic), and the dimethylacrylamide/2-acrylamido-2-methylpropane sulfonic acid may be “SU-Polymer 0-1” (manufactured by Toho Chemical Industry).

Polyacrylate crosspolymer-6, which is commercially available as “Sepimax Zen” from Seppic can be also used herein, especially in low pH environments.

The hydrophilic thickener in the water-based cosmetic may contain be a combination of one or more types.

### Emulsifiers

When the composition is in the form of an emulsion, it may contain an emulsifier. Emulsifiers may be nonionic, anionic, cationic, or zwitterionic. Some non-limiting examples of emulsifiers are disclosed in U.S. Pat. Nos. 3,755,560, 4,421,769, U.S. Publication No. 2006/0275237 and McCutcheon's Detergents and Emulsifiers, North American Edition, pages 317-324 (1986). Suitable examples of emulsifiers include non-ionic stearic acid derivative emulsifiers such as Polysorbate-20, glyceryl stearate, sorbitan stearate, PEG-100 stearate, Steareth-2, Steareth-21, glycereth-25 pyrrolidonecarboxylic acid isostearate and combinations of these.

### Other Optional Ingredients.

Compositions herein may include one or more optional ingredients known for use in topical cosmetic compositions, provided that the optional components do not unacceptably alter the desired benefits of the composition. In some instances, it may be desirable to select cosmetic actives that function via different biological pathways so that the actives do not interfere with one another. When the composition is in the form of an emulsion, the additional ingredients should not introduce instability into the emulsion (e.g., syneresis). For example, it may be desirable to select optional ingredients that do not form complexes with other ingredients in the composition, especially pH sensitive ingredients like vitamin B3 compounds, salicylates and peptides.

The additional ingredients should be suitable for use in contact with human skin tissue without undue toxicity, incompatibility, instability, allergic response, and the like. The optional components, when present, may be included at an amount of about 0.001% to 50% (e.g., 0.01% to 40%, 0.1% to 30%, 0.5% to 20%, or 1% to 10%), by weight of the composition. Some nonlimiting examples of additional ingredients include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunscreen agents, sunless tanning agents, lubricants, anti-acne agents, anti-cellulite agents, chelating agents, anti-wrinkle actives, anti-atrophy actives, phytosterols and/or plant hormones, N-acyl amino acid compounds, antimicrobials, and antifungals. Some particularly suitable examples of additional ingredient include one or more skin care actives selected from the group consisting of vitamin B3 compounds (e.g., niacinamide), n-acyl amino acids (e.g., undecylenoyl phenylalanine), vitamin E compounds (e.g., tocopheryl acetate), palmitoylated dipeptides (e.g., palmitoyl-lysine-threonine), palmitoylated pentapeptides (e.g., palmitoyl-lysine-threonine-threonine-lysine-serine), vitamin A compounds (e.g., retinol and retinyl propionate), and combinations thereof. Other non-limiting examples of optional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/0092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

Sucrose esters may be used herein. Such sucrose ester can be a blend of two or more sucrose esters, wherein the two or more sucrose esters are present at a ratio of any one sucrose ester to another of 1:10 to 1:1 (e.g., 1:7, 1:5, 1:3, or 1:2). In some instances, the sucrose ester may be a blend of sucrose laurate and sucrose dilaurate, wherein sucrose laurate is present at 50% to 80%, by weight of the sucrose ester, and the sucrose dilaurate is present at 20% to 45%, by weight of the sucrose ester. Alternatively, the sucrose ester may be a blend of sucrose laurate, sucrose dilaurate and sucrose trilaurate, wherein the sucrose dilaurate is present at 35% or more, by weight of the sucrose ester. A particularly suitable example of a sucrose ester for use herein is Sucrose Dilaurate BC10034 available from BASF.

### Method of Use of the Cosmetic Composition

The method of use herein includes identifying a target portion of skin on a person in need of treatment and applying the composition to the target portion of skin over the course of a treatment period. The target portion of skin may be on a facial skin surface such as the forehead, perioral, chin, periorbital, nose, and/or cheek) or another part of the body (e.g., hands, arms, legs, back, chest). The person in need of treatment is one whose skin exhibits signs of oxidative stress, such as fine lines, wrinkles, hyperpigmentation, uneven skin tone, and/or other visible skin features typically associated with aging. In some instances, the target portion of skin may not exhibit a visible sign of skin aging, but a user (e.g., a relatively young user) may still wish to target such an area of skin, if it is one that typically develops such issues as a person age. In this way, the present method may be used as a preventative measure to delay the onset of visible signs of skin aging.

The composition may be applied to a target portion of skin and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., a wrinkle or line) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied broadly to one or more skin surfaces. In certain embodiments, the compositions herein may be used as part of a multi-step beauty regimen, wherein the present composition may be applied before and/or after one or more other compositions.

### Combinations

1. A cosmetic composition, comprising by weight:
   a) from about 0.1% to about 10% of silica having an average particle size of from about 0.5 microns to about 35 microns, and having an oil absorbance of from about 1 ml/100 g to about 130 ml/100 g;
   b) from about 0.1% to about 10% of lauroyl lysine;
   c) from about 55% to about 99% of water.
2. The composition of the preceding feature, wherein the silica has a spherical shape.
3. The composition of any of the preceding features, wherein the silica has an average particle size of from about 1 micron to 3 lmicrons, alternatively from about 1 micron to 15 microns, alternatively from about 1 micron to about 10 microns.
4. The composition of any of the preceding features, wherein the lauroyl lysine has a flat polygonal shape.
5. The composition of any of the preceding features, wherein the lauroyl lysine has a mean particle size of from about 10 microns to about 40 microns, alternatively from about 15 microns to about 35 microns, alternatively from about 20 microns to about 30 microns.
6. The composition of any of the preceding features, wherein the silica has an oil absorbance of from about 1 ml/100 g to about 130 ml/100 g, alternatively from about 1 ml/100 g to about 120 ml/100 g, alternatively from about 1 ml/100 g to about 100 ml/100 g, alternatively from about 1 ml/100 g to about 50 ml/100 g.
7. The composition of any of the preceding features, wherein the weight ratio between silica and lauroyl lysine is from about 15:1 to about 1:10, alternatively from about 10:1 to about 1:10, alternatively from about 7:1 to about 1:7, alternatively from about 5:1 to about 1:5.
8. The composition of any of the preceding features, wherein the particle size ratio between silica and lauroyl lysine is from about 3:1 to about 1:35, alternatively from about 1:1 to about 1:35, alternatively from about 1:2 to about 1:25, alternatively from about 1:2 to about 1:20.
9. The composition of any of the preceding features, wherein when the composition comprises Shea butter, Cetyl alcohol, Stearyl alcohol and/or Behenyl alcohol, the total amount of Shea butter, Cetyl alcohol, Stearyl alcohol and Behenyl alcohol in the composition is up to about 0.5%, alternatively up to about 0.3%, alternatively up to about 0.1%.
10. Use of the composition of any of the preceding features, for providing: reduced heavy coated feel; reduced sticky/tacky feel; and/or reduced friction/draggy feel.
11. Use of the composition of any of the preceding features, for providing the reduction of the use of microplastic solid particulates, as an environmental benefit.

### EXAMPLES

Cosmetic compositions (wt %)

| Ingredient Name | Ex 1 | Ex 2 | Ex. 3 | CEx. i | CEx. ii |
|---|---|---|---|---|---|
| Water Purified | q.s | q.s | q.s | q.s | q.s |
| Glycerin | 5.500 | 5.500 | 5.500 | 5.500 | 5.500 |
| Xylitol | 1.000 | 1.000 | 1.000 | 1.000 | 1.000 |
| Niacinamide | 2.000 | 2.000 | 2.000 | 2.000 | 2.000 |
| pH buffer | 3.100 | 3.100 | 3.100 | 3.100 | 3.100 |
| Sepimax Zen *1 | 2.400 | 2.400 | 2.400 | 2.400 | 2.400 |
| Sepigel 305 *2 | 0.500 | 0.500 | 0.500 | 0.500 | 0.500 |
| Dimethicone 5 cst | 1.750 | 1.750 | 1.750 | 1.750 | 1.750 |
| Dimethicone 50 cst | 1.750 | 1.750 | 1.750 | 1.750 | 1.750 |
| DC1503 Fluid *3 | 1.500 | 1.500 | 1.500 | 1.500 | 1.500 |
| Glycereth-25 PCA Isostearate | 0.600 | 0.600 | 0.600 | 0.600 | 0.600 |
| Polysorbate 20 | 0.250 | 0.250 | 0.250 | 0.250 | 0.250 |
| Preservatives | 0.150 | 0.150 | 0.150 | 0.150 | 0.150 |
| Pentylene Glycol | 0.020 | 0.020 | 0.020 | 0.020 | 0.020 |
| Amihope LL *4 | 2.000 | 2.000 | 0.500 | X | 2.000 |
| Goddball E90C *5 | X | 1.500 | X | X | X |
| Goddball G6C *6 | 1.500 | X | 5.500 | X | X |
| Sunsil 150H *7 | X | X | X | X | 1.500 |
| Synthetic Mica and TiO2 *8 | 0.050 | 0.050 | 0.050 | 0.050 | 0.050 |
| Perfume | 0.040 | 0.040 | 0.040 | 0.040 | 0.040 |
| Total | 100.000 | 100.000 | 100.000 | 100.000 | 100.000 |
| Break time delta | 0.65 | 0.63 | 0.42 | −0.19 | 0.84 |
| Time weighted force area | 38594 | 36857 | 41516 | 49879 | 21629 |

| Ingredient Name | Ex. 4 | Ex. 5 | Ex. 6 | Ex. 7 | Ex. 8 |
|---|---|---|---|---|---|
| Water Purified | q.s | q.s | q.s | q.s | q.s |
| Glycerin | 5.500 | 10.000 | 5.500 | 3.000 | 7.000 |
| Xylitol | 1.000 | 1.000 | 1.000 | X | 1.500 |
| Trehalose | X | X | 3.000 | X | X |
| Butylene glycol | X | X | X | 5.500 | 5.000 |
| PEG/PPG/Polybutyleneglycol- | X | X | X | X | 2.000 |
| 8/5/3Glycerin | | | | | |
| PEG-32 | X | X | X | X | 2.000 |
| Niacinamide | 5.000 | 2.000 | 2.000 | 5.000 | 5.000 |
| pH buffer | 3.100 | 3.100 | 3.100 | 0.174 | 0.180 |
| Sepimax Zen *1 | 2.400 | 2.400 | 2.400 | X | X |
| Sepigel 305 *2 | 0.500 | 0.500 | 0.500 | 0.750 | 0.660 |
| Simulgel EG *9 | X | 0.500 | X | X | X |
| Ultrez 20 *10 | 0.260 | X | X | 0.290 | 0.300 |
| Pemulen TR-2 *11 | X | X | 0.150 | X | X |
| Xanthan gum | X | X | X | 0.050 | 0.050 |
| Caprylic/capric triglyceride | X | X | 1.000 | 0.500 | X |
| Eldew PS-203 *12 | X | X | 1.000 | 1.500 | X |
| Isohexadecane | X | X | 1.000 | X | X |
| Triethylhexanoin | X | X | 1.000 | 3.500 | X |
| Dimethicone 5 cst | 1.750 | 1.750 | 1.750 | X | X |
| Dimethicone 50 cst | 1.750 | 1.750 | 1.750 | X | X |
| DC1503 Fluid *3 | 1.500 | 1.500 | 1.500 | 1.500 | 1.500 |
| KF6011P *13 | X | X | X | X | 0.025 |
| Laureth 7 | X | X | X | 0.200 | X |
| Glycereth-25 PCA Isostearate | 0.600 | 0.600 | 0.600 | X | X |
| Polysorbate 20 | 0.250 | 0.250 | 0.250 | 0.500 | 0.250 |
| Preservative | 0.15 | 0.15 | 0.15 | 0.6 | 0.5 |
| Pentylene Glycol | 0.020 | 0.020 | 0.020 | 3.000 | 2.000 |
| Amihope LL *4 | 2.000 | 2.000 | 2.000 | 0.500 | 0.500 |
| Goddball G6C *5 | 1.500 | 1.500 | 1.500 | 2.000 | 1.500 |
| Synthetic Mica and TiO2 *8 | 0.500 | 0.050 | 0.050 | X | X |
| Natural MICA and TiO2 *14 | X | 0.500 | X | X | X |
| Boron nitride | 0.800 | X | X | 0.500 | X |
| Perfume | 0.040 | 0.040 | 0.040 | X | 0.020 |
| Total | 100.000 | 100.000 | 100.000 | 100.000 | 100.000 |

| | Ex. 9 | Ex. 10 | Ex. 11 |
|---|---|---|---|
| Water Purified | q.s. to 100% | q.s. to 100% | q.s. to 100% |
| Glycerin | 8.0 | 10.0 | 5.0 |
| Xylitol | X | 3.0 | 0.5 |
| Trehalose | 3.0 | X | 1.5 |
| Aminomethyl propanol | 1.3 | 1.3 | X |
| Sodium Benzoate | 0.05 | 0.05 | 0.05 |
| Disodium EDTA | 0.1 | 0.1 | 0.1 |
| Pentylene Glycol | 3.0 | 2.0 | X |
| 1,3-Butylene Glycol | 5.0 | 10.0 | X |
| Benzyl Alcohol | 0.4 | 0.2 | X |
| Triethylhexanoin | 5.0 | 10.0 | X |
| Caprylic Capric Triglyceride | 5.0 | X | 2.0 |
| Eldew PS-203 *12 | 1.5 | 5.0 | X |
| PMX-200 Silicone Fluid 5 cSt *15 | X | X | 3.5 |
| DC1503 *3 | X | X | 1.5 |
| Boron Nitride | X | X | 0.5 |
| Synthetic Mica and TiO₂ *8 | 2.0 | X | 0.5 |
| Natural Mica and TiO₂ *14 | X | 2.0 | X |
| Lauroyl Lysine *4 | 6.0 | 10.0 | 2.0 |
| Goddball G6C *5 | 1.0 | 1.0 | 3.0 |
| Polysorbate 20 | 0.5 | 1.0 | 0.25 |
| PEG-32 *15 | X | 1.0 | X |
| Sepigel 305 *2 | 0.6 | 1.0 | X |
| Ultrez 20 *10 | 0.25 | X | X |
| Pemulen TR-2 *11 | X | 0.15 | X |
| Sepimax Zen *1 | X | X | 1.2 |
| Xanthan gum | 0.05 | 0.1 | 0.1 |
| Niacinamide | 2.0 | 5.0 | 2.0 |
| Lactic Acid | X | X | 1.0 |
| Sodium Lactate | X | X | 1.0 |
| KSP-100 *17 | X | 1.5 | X |
| Phenoxyethanol | 0.20 | 0.10 | X |
| Perfume | 0.05 | 0.05 | 0.05 |

Details of the ingredients used in the above table

| | | |
|---|---|---|
| *1 | Sepimax Zen | INCI: Polyacrylate crosspolymer-6 |
| *2 | Sepigel 305 | Containing 40% of Polyacrylamide as active. INCI: |
| | | Polyacrylamide & Water & C13-14 Isoparaffin & Lauleth-7 |
| *3 | DC1503 | Dimethicone (5 cSt, Solvent) and Dimethiconol |
| *4 | Amihope LL | Lauroyl lysin available from Ajinomoto, having a flat hexagonal |
| | | shape and having a mean particle size of 20-30 microns |
| *5 | Goddball G6C | Silica having a mean particle size of 3-5 microns and an oil |
| | | absorbance of 30 ml/100 g, |
| *6 | Goddball E90C | Silica having a mean particle size of 30 microns and an oil |
| | | absorbance of 120 ml/100 g. |
| *7 | SUNSIL 150H | Silica having a mean particle size of 10-15 microns microns and |
| | | an oil absorbance of 140-210 ml/100 g), |
| *8 | Synthetic Mica and | Synthetic Mica layered with films of TiO₂, wherein the Synthetic |
| | TiO₂ | Mica has a mean particle size of 10-20 microns |
| *9 | Simulgel EG | INCI: Sodium Acrylate/Sodium Acryloyldimethyl Taurate |
| | | Copolymer & Isohexadecane & Polysorbate 80 |
| *10 | Ultrez 20 | INCI: Acrylates/C10-30 Alkyl Acrylate Crosspolymer |
| *11 | Pemulen TR-2 | INCI: Acrylates/C10-30 Alkyl Acrylate Crosspolymer |
| *12 | Eldew PS-203 | INCI: Phytosteryl/Octyldodecyl Lauroyl Glutamate |
| *13 | KF6011P | PBG-11 METHYL ETHER DIMETHICONE |
| *14 | Natural Mica and | Natural Mica layered with films of TiO₂, wherein the Natural |
| | TiO₂ | Mica has a mean particle size of 10-20 microns |
| *15 | PMX-200 Silicone | Dimethicone 5 cSt |
| | Fluid 5 cSt | |
| *16 | PEG-32 | having MW of 1540 |
| *17 | KSP-100 | INCI: Vinyl Dimethicone/Methicone Silsesquioxane |
| | | Crosspolymer |

### Tackiness (Break Time Delta)

Tackiness is evaluated by Break time delta, which is measured by below “Tack method”. Higher value of Break time delta means reduced persistent stickiness.

### Moisturized feel (Time Weighted Force Area)

Moisturized feel is evaluated by Time weighted force area, which is measured by below “Tack method”. Higher value of Time weighted force area means higher moisturized feel.

### Tack Method (Also Called as Tack Test)

The method uses a texture analyzer to contact a probe with a film formed from the composition. The texture analyzer then measures the force needed to separate the probe from the composition film. Tack Force, Time Weighted Force Area, Mean Break Time and the change in Break Time can be determined by this method. The Tack Method is configured to run for a period of 100 minutes. It is believed, without being limited by theory, that the tackiness properties exhibited by the composition over the course of the Tack Test approximate the tackiness properties exhibited by the composition during the first few minutes (e.g., less than 10 minutes, between 30 seconds and 5 minutes, or between 1 minute and 3 minutes) of use of the composition by a user. It is also believed, without being limited by theory, that having a suitable tack force at the later time periods in the test (e.g., 60 minutes, 80 minutes, and/or 100 minutes), influences the moisturization perception of the user more than the tack force at earlier time periods in the test. Thus, time weighted force area is used to weight the tack force measurements toward the later time points, and thereby provide a more accurate prediction of the moisturisation signal perceived by a user.

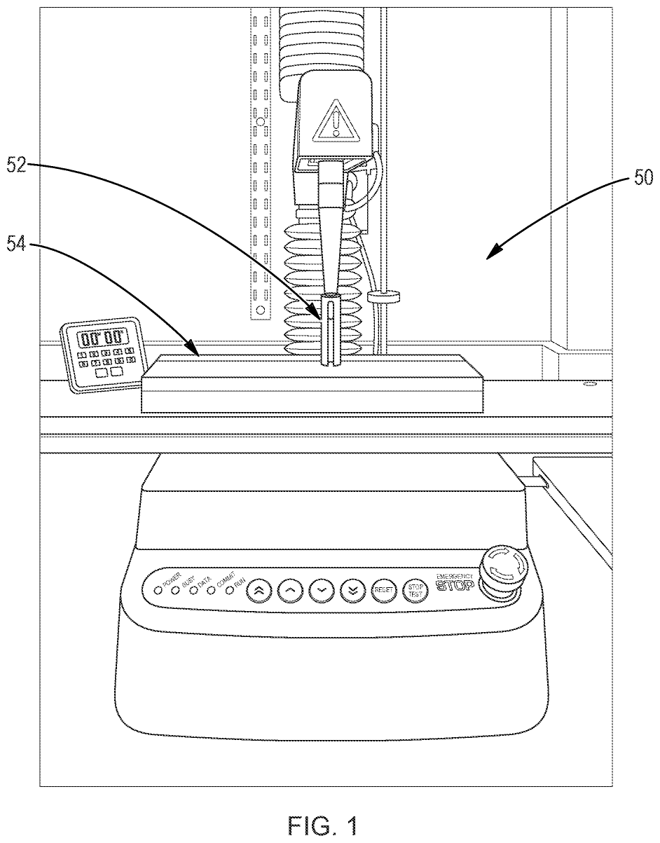

FIG. 1 illustrates an exemplary test setup for the Tack Test. A TA.XT2i brand texture analyzer (available from Texture Technologies Corporation, MA) 50 or equivalent is used to measure the tack of a film formed from a composition of interest. The texture analyzer is equipped with a tack probe 52 in the form of a 12.75 mm diameter acrylic cylinder with a flat surface. During testing, the flat surface of the probe 52 is contacted with the surface of a film of the composition. Thus, the flat surface of the probe and the surface of the film must be parallel to one another during testing to ensure there is sufficient contact by the film across the effective test surface of the probe 52. The film is made by filling or overfilling (followed by draw down) a rectangular channel 54 (e.g., 25 cm long×30 mm wide a 0.25 mm deep) with the composition to be tested.

The test is conducted using an adhesive test protocol with a pretest speed of 0.10 mm/second, a test speed of 0.10 mm sec and a post-test speed of 1.0 mm/sec. The applied force is 200 g, the return distance is 4 mm and the contact time is 5.0 sec. The trigger type to designate sample contact is set to automatic and the trigger force is 5.0 g. The test is run and at the following time increments immediately after the film is prepared: <1 minute (i.e., immediately following preparation of the film), 10, 20, 30, 40, 50, 60, 80, and 100 minutes. Each time point is run on a previously undisturbed/untested area of the sample. Each sample is run in triplicate and the averages are recorded.

Data extraction uses the portion of data that is collected as the probe pulls upward out of the sample. Tack force is the peak force of each test run. Time weighted force area is determined by the following formula:

$$time weighted force area = ( t 1 - t 2 ) * [ \frac{P 1 - P 2}{200} ] * [ \frac{t 1 + t 2}{2} ]$$

### Where:

t1=the later of the two times in the time range at which the measurement was taken;
t2=the earlier of the two times in the time range at which the measurement was taken;
P1=peak force in grams at time 1 in the pair of times being calculated; and
P2=peak force in grams at time 2 in the pair of times being calculated.

When calculating the difference between t1 and t2 or P1 and P2, the absolute value of the result is used to calculate time weighted force area.

Time Weighted Force Area is reported as the sum of the individual time weighted force area values calculated for each of the 8 times intervals (<1 min to 10 min, 10 min to 20 min, 20 min to 30 min, etc.).

Break time is determined by the width of the force curve. The start time of the Break time calculation is when the sign of the force exerted by the texture analyzer changes to positive from negative at time zero (i.e., the start of the test) and the time at which the force decays back to 0.0+/−0.02 from the peak force as the break time. Mean Break Time is reported as the average of the break times at 60, 80 and 100 minutes. The change in break time is the difference between the initial break time and the break time at 100 minutes.

The results of some of the above compositions are shown above, together with the composition details.

Ex. 1 through Ex. 11 are the inventive examples, which are suitably used as oil-in-water emulsion cosmetic composition, in a form of essence, lotion, serum and/or gel cream. The examples can provide improved sensory feel during and/or after the application to skin, such as: reduced heavy coated feel; reduced sticky/tacky feel; and/or reduced friction/draggy feel, while providing moisturized feel, especially in cosmetic compositions containing a higher level of water, and/or in the cosmetic compositions containing lower level of oils.

The benefit, especially reduced tacky feel while providing moisturized feel, can be also understood by the comparison of Ex. 1-3 to comparative examples, i.e., CEx. i which does not contain lauroyl lysine and silica and which provides tacky feel. CEx. ii in which silica has a higher oil absorbance and which provide reduced moisturized feel.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic composition, comprising by weight:
a) from about 0.1% to about 10% of a silica comprising an average particle size of from about 0.5 microns to about 35 microns and an oil absorbance of from about 1 ml/100g to about 130 ml/100 g;
b) from about 0.1% to about 10% of a lauroyl lysine;
c) from about 55% to about 99% of water,

2. The composition of claim 1, wherein the silica has a spherical shape.

3. The composition of claim 2, wherein the average particle size of the silica is from about 1 micron to 15 microns.

4. The composition of claim 4, wherein the average particle size of the silica is from about 1 micron to about 10 microns.

5. The composition of claim 1, wherein the lauroyl lysine comprises a flat polygonal shape.

6. The composition of claim 1, wherein the lauroyl lysine comprises a mean particle size of from about 10 microns to about 40 microns.

7. The composition of claim 6, wherein the mean particle size of the lauroyl lysine is from about 20 microns to about 30 microns.

8. The composition of claim 1, wherein the oil absorbance of the silica is from about 1 ml/100 g to about 100 ml/100 g.

9. The composition of claim 8, wherein the oil absorbance of the silica is from about 1 ml/100 g to about 50 ml/100 g.

10. The composition of claim 1, wherein the weight ratio between the silica and the lauroyl lysine is from about 15:1 to about 1:10.

11. The composition of claim 10, wherein the weight ratio between the silica and the lauroyl lysine is from about 10:1 to about 1:10.

12. The composition of claim 11, wherein the weight ratio between the silica and the lauroyl lysine is from about 7:1 to about 1:7.

13. The composition of claim 12, wherein the weight ratio between the silica and the lauroyl lysine is from about 5:1 to about 1:5.

14. The composition of claim 1, wherein the particle size ratio between the silica and the lauroyl lysine is from about 3:1 to about 1:35.

15. The composition of claim 14, wherein the particle size ratio between the silica and the lauroyl lysine is from about 1:1 to about 1:35.

16. The composition of claim 15, wherein the particle size ratio between the silica and the lauroyl lysine is from about 1:2 to about 1:25.

17. The composition of claim 16, wherein the particle size ratio between the silica and the lauroyl lysine is from about 1:2 to about 1:20.

18. The composition of claim 1, wherein when the composition comprises up to about 0.5% total of the combination of shea butter, cetyl alcohol, stearyl alcohol, and/or behenyl alcohol.

19. The composition of claim 18, wherein when the composition comprises up to about 0.3% total of the combination of shea butter, cetyl alcohol, stearyl alcohol, and/or behenyl alcohol.

20. The composition of claim 19, wherein when the composition comprises up to about 0.1% total of the combination of shea butter, cetyl alcohol, stearyl alcohol, and/or behenyl alcohol.
